(19) United States
(12) Patent Application Publication
(10) Pub. No.: US 2004/0249430 A1
(43) Pub. Date: Dec. 9, 2004

(54) Implantable medical electrical lead

(75) Inventors: Gonzalo Martinez, Mendota Heights, MN (US); Kathryn R. Parsons, Fridley, MN (US); Bret R. Shoberg, Corcoran, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/453,707

(22) Filed: Jun. 3, 2003

(51) Int. Cl. A61N 1/05

(52) U.S. Cl. 607/122

(57) ABSTRACT

A medical lead includes a lead body and an electrode formed by a plurality of coil turns and positioned about the lead body. A portion of a conductor extends from an internal lumen of the lead body, through an opening, to an external surface of the lead body, to electrically couple with the electrode; the portion of the conductor is press fit between at least one of the plurality of turns of the electrode and the lead body thereby making direct electrical contact with the electrode.

### TECHNICAL FIELD OF THE INVENTION

This invention relates generally to electrical leads for use with implantable medical devices, and more particularly to electrical coupling between a conductor and an electrode of such medical leads.

### BACKGROUND OF THE INVENTION

Implantable defibrillation leads typically include an electrode in the form of a high-voltage defibrillation coil. This coil is electrically coupled to an associated high-voltage conductor coil or cable that extends through the lead body. Pacing leads, cardiac catheters, muscle stimulation leads, and other electrode bearing leads may also employ similar coil/conductor arrangements.

Many techniques such as welding, crimping, brazing, swaging, etc., have been proposed/utilized for producing the necessary electrical interconnections between the defibrillation coil and the high-voltage conductor. Obviously, such interconnections must be reliable and possess desirable mechanical properties. For example, U.S. Pat. No. 5,676,694 entitled “Medical Electrical Lead”, issued Oct. 14, 1997 and assigned to the assignee of the present invention, the teachings of which are hereby incorporated by reference, discloses a connection mechanism wherein the coil is electrically coupled to the high-voltage conductor by means of a conductive crimp-sleeve fabricated from a conductive biocompatible metal such as titanium, stainless steel, tantalum or platinum. A portion of the conductor insulation is stripped away and inserted into a longitudinal lumen through a tubular portion of the crimp-sleeve. The tubular portion is attached to the conductor by means of a plurality of crimps spaced around the circumference of the tubular portion. An end portion of the coil is welded or brazed to the sleeve to provide electrical coupling between the coil and the conductor.

Traditional means employed to couple electrodes to conductors, such as those described in the above-cited patent, require additional components (i.e. crimping/weld sleeves) and/or manufacturing steps (e.g. crimping, welding, etc.), whereas embodiments of the present invention accomplish direct electrical coupling between a conductor and an electrode.

### BRIEF DESCRIPTION OF THE DRAWINGS

The following drawings are illustrative of particular embodiments of the invention and therefore do not limit its scope, but are presented to assist in providing a proper understanding of the invention. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a plan view of an implantable lead assembly in accordance with an embodiment of the present invention;

FIG. 2 is a radial cross-sectional view of the lead assembly shown in FIG. 1 taken along line 2-2;

FIG. 3 is an axial cross-sectional view of a portion of a lead assembly in accordance with another embodiment of the present invention;

FIG. 4 is a plan view of the embodiment shown in FIG. 3;

FIG. 5A is an axial cross-sectional view of a portion of a lead assembly in accordance with yet another embodiment of the present invention;

FIG. 5B is an axial cross-sectional view of a portion of a lead assembly in accordance with yet another embodiment of the present invention;

FIG. 6 is a plan view of the embodiment shown in FIG. 5A;

FIG. 7 is a radial cross-sectional view illustrating a first alternative configuration of the embodiment shown in FIGS. 5A-B and FIG. 6;

FIG. 8 is a radial cross-sectional view illustrating a second alternative configuration of the embodiment shown in FIGS. 5A-B and FIG. 6;

FIG. 9 is an axial cross-sectional view of a portion of a lead assembly in accordance with yet another embodiment of the present invention; and

FIG. 10 is a plan view of the embodiment shown in FIG. 9.

### DETAILED DESCRIPTION OF THE INVENTION

The following description is exemplary in nature and is not intended to limit the scope, applicability, or configuration of the invention in any way. Rather, the following description provides a convenient illustration for implementing exemplary embodiments of the invention. Various changes to the described embodiments may be made in the function and arrangement of the elements described herein without departing from the scope of the invention.

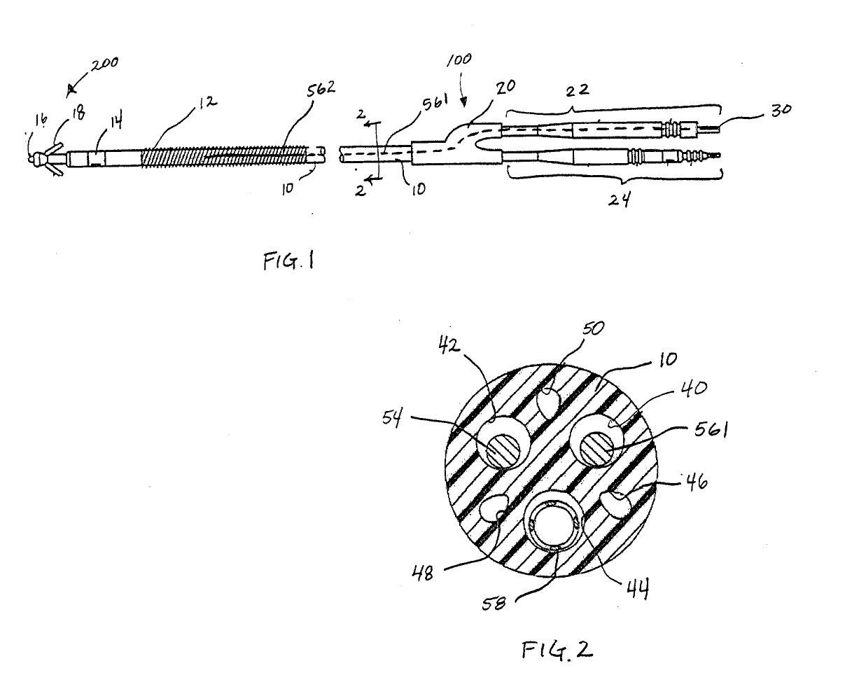

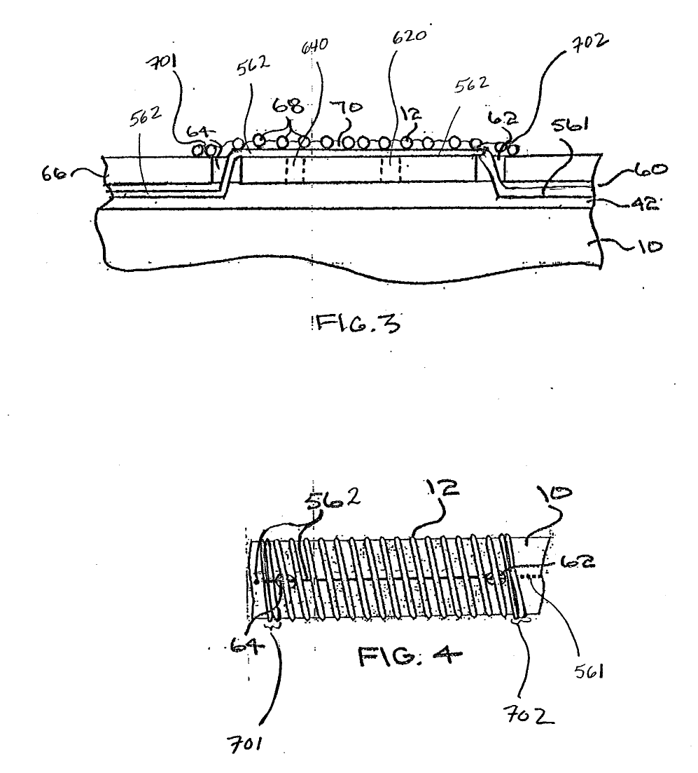

FIG. 1 is a plan view of an implantable lead assembly in accordance with an embodiment of the present invention. The lead of FIG. 1 includes an elongated insulative lead body 10 fabricated, for example, of silicone rubber, polyurethane or other biocompatible elastomer. At a distal end 200 of the lead, there is carried an elongated defibrillation coil 12, a ring electrode 14, and a tip electrode 16. Tines 18 are provided to maintain electrode 16 in contact with the tissue of the right ventricle. Alternatively, a helix tip electrode, such as is commonly known to those skilled in the art, may be employed in place of tip electrode 16 and tines 18. Electrodes 14 and 16 correspond to any conventionally available pacing and sensing electrodes and are coupled to connector contacts, carried in a bipolar connector leg 24 extending from a bifurcation 20 at a proximal end 100 of lead body 10, via elongated conductors 54 and 58 (FIG. 2), respectively. Likewise, electrode 12 corresponds to any conventionally available defibrillation electrode and is coupled to a connector pin 30, carried by a unipolar connector leg 22 extending from bifurcation 20 at proximal end 100 of lead body 10, via an elongated conductor including a first conductor portion 561 extending within lead body 10 and a second conductor portion 562 extending between electrode 12 and lead body 10. According to embodiments of the present invention, first conductor portion 561 coupled to connector pin 30 extends to second conductor portion 562, which exits lead body 10 to electrically couple with electrode 12, being held between electrode 12 and lead body 10. Although FIG. 1 illustrates second conductor portion 562 extending beneath a plurality of coil turns of electrode 12, the present invention allows alternative configurations wherein second portion 562 is held beneath a single turn of electrode 12. Furthermore, in alternate embodiments, several coil turns of electrode 12, at each end of electrode 12, may be welded together.

FIG. 2 is a radial cross-sectional view of the lead assembly shown in FIG. 1 taken along line 2-2, wherein lead body 10 is illustrated as a multi-lumen lead body of the type shown and described in U.S. Pat. No. 5,584,873 entitled “Medical Lead Compression Lumens”, issued Dec. 17, 1996 and assigned to the assignee of the present invention, the teachings of which are hereby incorporated by reference. As illustrated in FIG. 2, lead body 10 includes conductor lumens 40, 42, and 44 carrying conductors 561(first portion), 54 and 58, respectively, wherein conductors 561 and 54 are in the form of generally straight, bundled stranded wire cables and conductor 56 is in the form of a coiled conductor. Wires forming conductors 561, 54 and 56 may be made of MP35-N alloy, well known in the art, or any other biostable and biocompatible material that is capable of reliably conducting electrical current after having been subjected to numerous, repeated bending and torsional stresses. FIG. 2 further illustrates lead body 10 including compression lumens 46, 48, and 50, as taught in the '873 patent, which are not necessary elements according to embodiments of the present invention.

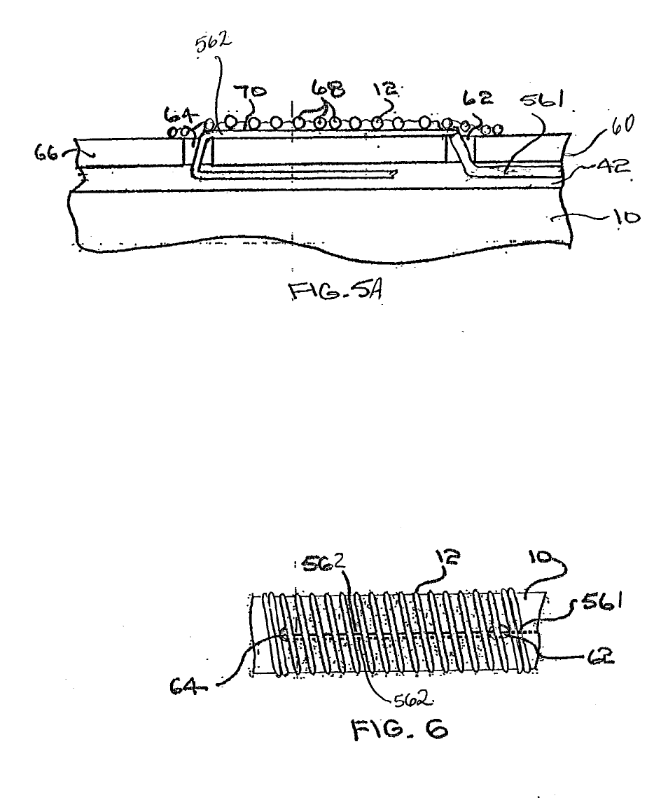

FIG. 3 and FIG. 4 are cross-sectional and plan views respectively of a portion of an implantable lead assembly in accordance with an embodiment and of the present invention. FIG. 3 illustrates first conductor portion 561 extending distally within lumen 42 to a first opening 62 in proximity to a proximal end 60 of the lead assembly portion shown; second conductor portion 562 extends out first opening 62 to be positioned between lead body 10 and coil electrode 12 and then re-enters lumen 42 via a second opening 64 positioned in proximity to a distal end 66 of the lead assembly portion shown. Embodiments of the present invention include first opening 62 and second opening 64 positioned as illustrated in FIG. 4 but also include alternate positions of openings 620, 640 as indicated by dashed lines located more toward a middle portion of electrode 12. Referring to FIG. 4, first conductor portion 561 is shown as a dotted line as it passes through lumen 42 toward first opening 62 and second conductor portion 562 as a solid line residing between the lead body 10 and electrode 12.

According to embodiments of the present invention, second conductor portion 562 is press fit in position under the force of the windings of electrode 12, thus maintaining second conductor portion 562 in electrical contact with one or more of the plurality of windings 68. Furthermore, according to one embodiment, turns of electrode 12 at one or both ends 701, 702 are joined together to help maintain the press fit of second conductor portion 562; examples of joining means include but are not limited to welding. Of course, second conductor portion 562 must be free of insulation to effectuate the desired electrical contact, while first conductor portion 561 may have a layer of insulation formed thereover. Electrode 12 is made of any low resistance, corrosion resistant material that is biocompatible and biostable, examples of such materials include platinum/iridium alloys, and second conductor portion 562 residing between lead-body 10 and electrode 12 may be polished as for example by electro-polishing, mechanical polishing, or chemical polishing, to reduce wear due to friction at an interface between second conductor portion 562 and electrode 12. Second conductor portion 562 may also be coated with a layer of smooth metal (e.g. gold, platinum, etc.). As further illustrated in FIG. 3, according to some embodiments of the present invention, a filler material 70 is deposited between coil turns of electrode 12 and over second conductor portion 562. Filler material 70 according to some embodiments, is adapted to prevent significant relative motion between electrode 12 and second conductor portion 562 by anchoring second conductor portion 562 and/or electrode to lead body 10, while, according to alternate embodiments, filler material 70 is adapted to simply encapsulate portions of second conductor portion 562 in order to prevent corrosion of second conductor portion 562; in either type of embodiment filler material 70 may be an polymer adhesive, such as a silicone medical adhesive or a polyurethane adhesive, both known to those skilled in the art of lead construction. Furthermore, according to various embodiments, filler material 70 includes a conductive material so that material 70 may be present in between second conductor portion 562 and electrode 12 without significantly impairing electrical coupling between the two. FIG. 3 also illustrates filler material 70 extending into first opening 62 and second opening 64 according to some embodiments of the present invention wherein filler material 70 further serves to seal off lumen 42 and/or anchor second conductor portion 562 to lead body by filling in openings 62, 64 around conductor 561, 562; according to alternate embodiments a separate material from filler material 70 is used to fill in openings 62, 64.

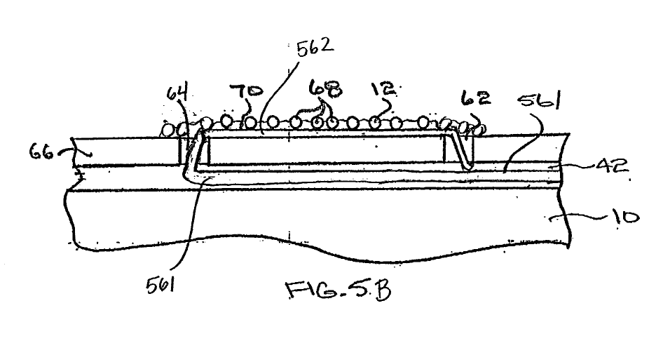

FIG. 5A is an axial cross-sectional view of a portion of a lead assembly and FIG. 6 is a plan view of the portion shown in FIG. 5A. As illustrated in FIG. 5A, according to another embodiment of the present invention, first conductor portion 561 extends distally within lumen 42 to a first opening 62 and second conductor portion 562 re-enters lumen 42, through second opening 64, and extends proximally, toward proximal end 60 of the lead assembly portion shown. Referring to FIG. 6, first conductor portion 561 is shown as a dotted line as it passes through lumen 42 toward first opening 62 and second conductor portion 562 as a solid line residing between the lead body 10 and electrode 12 and a dashed line within lumen 42 extending back from second opening 64 toward first opening 62. FIG. 5B is an axial cross-sectional view of a portion of a lead assembly according to yet another embodiment of the present invention wherein first conductor portion 561 extends within lumen 42 toward second opening 64 in proximity to distal end 66 where it exits lumen 42 as second conductor portion 562, which extends proximally between electrode 12 and lead body 10 to re-enter lumen 42 through first opening 62. According to the embodiment illustrated in FIG. 5B, routing of second conductor portion from second opening 64, in proximity to distal end 66, proximally to first opening 62 may facilitate an assembly of electrode 12 from a distal end of lead body 10 onto lead body 10 over second conductor portion 562 after second conductor portion 562 has been assembled into lead body 10.

Although FIGS. 3-5B illustrate embodiments including first and second openings 62, 64, it should be recognized that as illustrated in part in FIG. 1, second conductor portion 562 need not re-enter lead body 10 through a second opening, but may be terminated anywhere beneath electrode 12 according to embodiments of the present invention.

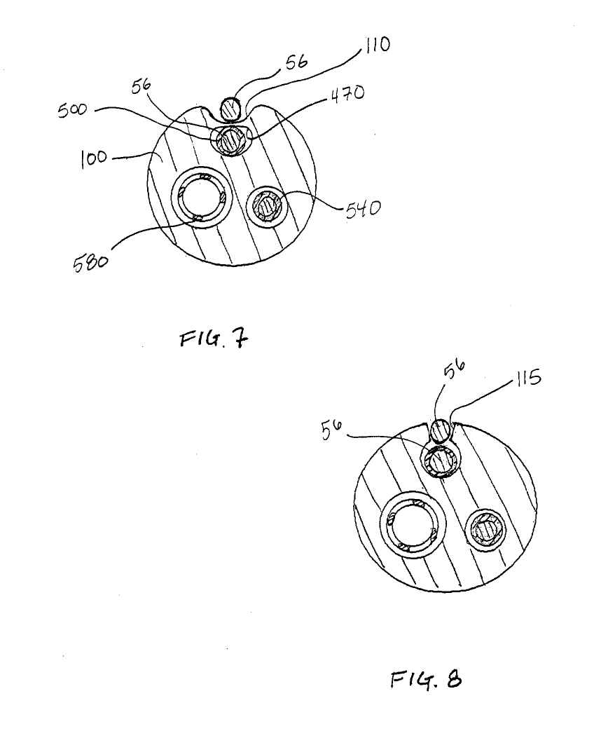

FIG. 7 is a radial cross-sectional view illustrating a first alternative configuration of the embodiments shown in FIGS. 5A-B and FIG. 6. FIG. 7 illustrates a conductor 56 assembled into a lead body 100, wherein a first portion of conductor 56, including a layer of insulation 500 formed thereover, resides in lumen 42 and a second portion of conductor 56, stripped of insulation, resides within a longitudinal channel 110 formed on an exterior surface of lead body 100. According to some embodiments of the present invention longitudinal channel 110 is preformed into lead-body 100 to accommodated second portion of conductor 56, however in alternate embodiments, a dedicated channel of this nature is not required since the windings of coil 12 (FIGS. 1-5B) passing over second portion of conductor 56 exert a radially inward force thereon and cause resilient lead-body 100 to become somewhat deformed into, for example, the shape shown in FIG. 7, wherein this resilient force maintains conductor 56 in contact with turns of electrode 12. FIG. 8 is a radial cross-sectional view illustrating a second alternative configuration of the embodiment shown in FIGS. 5A-B and FIG. 6. FIG. 8 illustrates a longitudinal opening 115, as an alternative to channel 110 illustrated in FIG. 7, which accommodates second portion of conductor 56. According to some embodiments, an adhesive backfill is employed to anchor conductor 56 in channel 115.

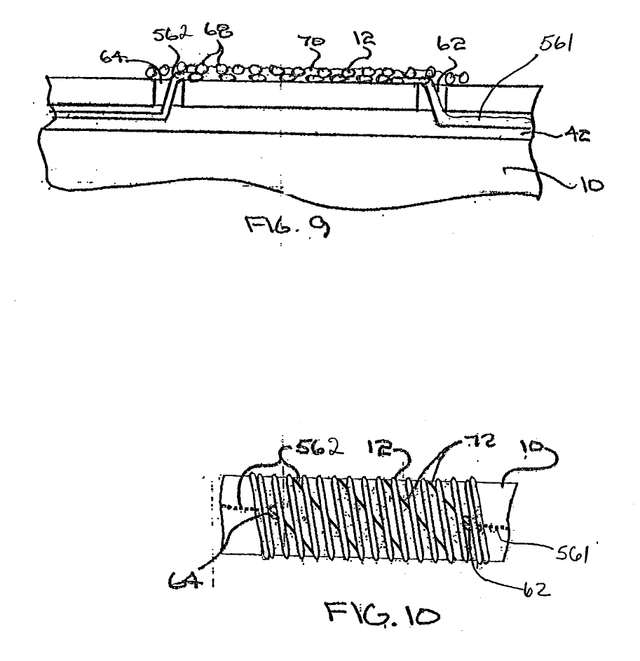

FIG. 9 is an axial cross-sectional view of a portion of a lead assembly in accordance with yet another embodiment of the present invention; and FIG. 10 is a plan view of the embodiment shown in FIG. 9. The embodiment illustrated in FIGS. 9 and 10 differs from previously described embodiments in that second conductor portion 562 is wound around lead-body 10 beneath electrode 12 as is shown at 72. Although first conductor portion 561 is illustrated exiting lumen 42 at first opening 62 and second conductor portion 562 re-entering lumen 42 at second opening 64 where second conductor portion 562 extends distally, alternate embodiments of the present invention include second conductor portion 562 extending proximally upon re-entry into lumen 42 and first conductor portion 561 exiting at second opening 64 and second conductor portion 562 re-entering at first opening 62. Furthermore, it should be recognized that second conductor portion 562 need not re-enter lead body 10 through a second opening, but may be terminated anywhere beneath electrode 12 according to embodiments of the present invention

In the foregoing specification, the invention has been described with reference to specific embodiments. However, it may be appreciated that various modifications and changes can be made without departing from the scope of the invention as set forth in the appended claims. For example, an electrode described herein as a coil may further include a non-coiled portion, for example a solid ring, which may make additional electrical contact with a conductor electrically coupled to the electrode by means of the present invention. Accordingly, the specification and figures are to be regarded as illustrative rather than as restrictive, and all such modifications are intended to be included within the scope of the present invention.

What is claimed is:

1. A medical electrical lead, comprising:
an elongated lead body including an external surface and an internal longitudinally extending lumen;
an electrode formed by a plurality of coil turns and positioned about the external surface of the lead body;
a first opening formed between the lumen and the external surface of the lead body; and
a conductor including a first portion extending within the lumen and exiting the lumen through the first opening and a second portion extending out from the first opening and electrically coupled to the electrode via a press fit between at least one of the plurality of coil turns of the electrode and the lumen of the lead body.

2. The medical electrical lead of claim 1, wherein the electrode includes a first end and a second end and wherein two or more of the plurality of coil turns located at the first end and at the second end are joined together.

3. The medical electrical lead of claim 1, wherein the second portion of the conductor is held against the external surface of the lead body between the at least one of the plurality of coil turns and the lumen.

4. The medical electrical lead of claim 1, further comprising a filler material deposited over the second portion of the conductor and between a portion of the plurality of coil turns in proximity to the second portion of the conductor.

5. The medical electrical lead of claim 4, wherein the filler material is conductive.

6. The medical electrical lead of claim 4, wherein the filler material is an adhesive.

7. The medical electrical lead of claim 4, wherein the filler material prevents corrosion of the second portion of the conductor.

8. The medical electrical lead of claim 4, wherein the filler material prevents significant relative motion between the at least one of the plurality of coil turns and the conductor.

9. The medical electrical lead of claim 1, wherein the second portion of the conductor is substantially aligned with the lumen.

10. The medical electrical lead of claim 1, wherein the second portion of the conductor winds about the external surface of the lead body.

11. The medical electrical lead of claim 1, wherein a longitudinal channel is formed in the external surface of the lead body to accommodate the second portion of the conductor.

12. The medical electrical lead of claim 1, further comprising:
a second opening formed between the lumen and the external surface of the lead body; the second opening longitudinally spaced from the first opening;
wherein the second portion of the conductor enters the lumen through the second opening.

13. The medical electrical lead of claim 12, wherein the electrode includes a first end and a second end and wherein the first opening and the second opening are positioned in proximity to the first end and the second end.

14. The medical electrical lead of claim 12, wherein the electrode includes a first end and a second end and wherein the first opening and the second opening are positioned away from the first end and the second end and toward a point centered between the first end and the second end.

15. The medical electrical lead of claim 12, wherein the second opening is positioned proximally to the first opening.

16. The medical electrical lead of claim 12, wherein the second opening is positioned distally to the first opening.

17. The medical electrical lead of claim 12, wherein the second portion of the conductor extends distally from the second opening through the lumen.

18. The medical electrical lead of claim 12, wherein the second portion of the conductor extends proximally from the second opening through the lumen.

19. A medical electrical lead according to claim 1 wherein the second portion of the conductor includes a wear-resistant and corrosion-resistant coating.

20. A medical electrical lead, comprising:
an elongated lead body including an external surface and an internal longitudinally extending lumen;
an electrode formed by a plurality of coil turns and positioned about the external surface of the lead body;
a first opening formed between the lumen and the external surface of the lead body;
a second opening formed between the lumen and the external surface of the lead body and positioned proximally to the first opening; and
a conductor including a first portion extending within the lumen and exiting the lumen through the first opening and a second portion extending from the first opening, electrically coupled with the electrode coil, and entering the lumen through the second opening;
wherein the second portion of the conductor is held between at least one of the plurality of coil turns and the lumen.

21. The medical electrical lead of claim 20, further comprising means to prevent corrosion of the second portion of the conductor.

22. The medical electrical lead of claim 20, further comprising means to prevent significant relative motion between the at least one of the plurality of coil turns and the conductor.

23. The medical electrical lead of claim 20, wherein the electrode includes a first end and a second end and wherein two or more of the plurality of coil turns located at the first end and at the second end are joined together.

24. The medical electrical lead of claim 20, wherein the second portion of the conductor is substantially aligned with the lumen.

25. The medical electrical lead of claim 20, wherein the second portion of the conductor winds about the external surface of the lead body.

26. A method for electrically coupling a conductor to an electrode in a medical electrical lead comprising:
passing a portion of the conductor from inside a body of the lead to outside the body; and
positioning coil turns of the electrode about the body of the lead to press fit the portion of the conductor against the body of the lead and thereby make direct electrical contact with the conductor.

27. The method of claim 26, further comprising depositing a filler material over the portion of the conductor and between the coil turns of the electrode.
